(19) United States
(12) Patent Application Publication
(10) Pub. No.: US 2025/0066288 A1
(43) Pub. Date: Feb. 27, 2025

(54) DIHYDRAZIDE-DIHYDRAZONE COMPOUNDS WITH ADAMANTYL MOIETY AND A PROCESS FOR SYNTHESIS OF THE SAME

(71) Applicant: Rupa Sachin Pawar, Pune (IN)

(72) Inventor: Rupa Sachin Pawar, Pune (IN)

(21) Appl. No.: 18/723,923

(22) PCT Filed: Dec. 30, 2022
(86) PCT No.: PCT/IB2022/062912

§ 371 Date: Jun. 25, 2024

(30) Foreign Application Priority Data
Jun. 11, 2022 (IN) 202221033544

(51) Int. Cl. C07C 251/80; A61K 31/165; A61K 31/341; A61K 31/381; C07D 307/70; C07D 333/24

(52) U.S. Cl. CPC C07C 251/80; A61K 31/165; A61K 31/341; A61K 31/381; C07D 307/70; C07D 333/24; C07C 2601/16; C07C 2602/10; C07C 2603/86

(57) ABSTRACT

The present disclosure relates generally to organic compounds. More specifically, the disclosure is directed to a dihydrazide-dihydrazone compound of Formula (I), a stereoisomer, a tautomer, a pharmaceutically acceptable salt or a pharmaceutically acceptable solvate thereof. The compounds of the present disclosure possess anti-microbial activity, specifically antifungal and antibacterial activity. The present disclosure also provides a process of synthesis of the compound of Formula (I).

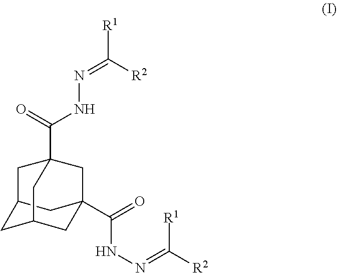

### FIELD OF THE INVENTION

The present disclosure relates generally to organic compounds. More specifically, the disclosure is directed to a dihydrazide-dihydrazone compound of Formula I, a stereoisomer, a tautomer, a pharmaceutically acceptable salt or a pharmaceutically acceptable solvate thereof. The compounds of the present disclosure possess anti-microbial activity. The present disclosure also provides a process of synthesis of the compound of Formula I.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

Microbes are unicellular microscopic living organisms that are present all around us. They co-exist with other organisms in their natural environment which includes the human gut. Ordinarily most microbes belong to one of the classes—viruses, bacteria, fungi, archaea, or protozoa. Each class of these microbes possesses pathogenic microbes that are disease causing, for example Staphylococcus sp., Enterovirus, Candida sp. etc. Pathogenic microbes can wreak havoc as was seen in coronavirus pandemic of 2019 and therefore their control is very important.

Multi-pronged measures are required for controlling the spread and growth of microbes. This includes implementing public health policies, strict measures of quarantining or social distancing, serological screening, and effective treatment of the microbes. The most important of these is the effective treatment of microbes by medicines or drugs which can curb or slow down the growth of the organism, or kill them. Many drugs exist in the market however a number of them have been rendered ineffective due to development of antibiotic resistance. Antibiotic resistance refers to a modification or evolution in the genome of the microbes that makes them immune to antimicrobials. Thus development of novel active compounds that can target the microbes effectively and against which resistance has not developed are essential. Unfortunately, the development of antimicrobials is a tedious process and has further slowed down in the last few decades leading to lesser available new compounds to counter the surge in antimicrobial resistance. Thus, there is an urgent need in the art to develop new antimicrobial compounds.

The inventors of the present disclosure provide compounds with high antimicrobial efficacy and methods of their synthesis.

### OBJECTS OF THE INVENTION

An object of the present disclosure is to provide novel antimicrobial compounds of Formula I.

Another object of the present disclosure is to provide a process of synthesis of the antimicrobial compounds.

### SUMMARY OF THE INVENTION

This summary is provided to introduce a selection of concepts in a simplified form that are further described below in Detailed Description section. This summary is not intended to identify key features or essential features of the claimed subject matter, nor is it intended to be used as an aid in determining the scope of the claimed subject matter.

Aspects of the present disclosure provide a series of dihydrazide-dihydrazone compounds with adamantyl moiety with potential antimicrobial activity.

In an aspect, the present disclosure provides a compound of Formula I, a stereoisomer, a tautomer, a pharmaceutically acceptable salt or a pharmaceutically acceptable solvate thereof,

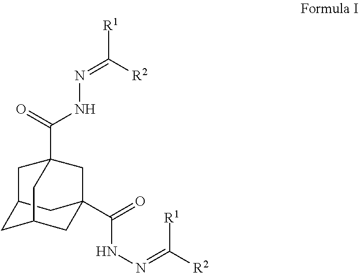

wherein R¹ is an unsubstituted or substituted C₆₋₁₀ aryl or an unsubstituted or substituted C₄₋₁₀ heterocyclyl;
R² is selected from H, C₁₋₆alkyl, C₁₋₆alkoxy, halogen, nitro, —COOH or combinations thereof; and wherein the substituents are selected from one or more of halogen, nitro, C₁₋₆alkyl, C₁₋₆alkoxy, —COOH, hydroxy, cyano, or NH₂.

In an embodiment, the compound of Formula I may be selected from:
Bis [N′-(4-nitro)benzylidene]adamantane-1,3-dicarbohydrazide;
Bis [N′-(3-methoxy, 4-hydroxy)benzylidene]adamantane-1,3-dicarbohydrazide;
Bis [N′-(1-(2-hydroxy, 5-methyl phenyl)ethylidene)]adamantane-1,3-dicarbohydrazide;
Bis [N′-(3,4-dimethoxy)benzylidene)]adamantane-1,3-dicarbohydrazide;
Bis [N′-(1-(4-ethoxy)ethylidene)]adamantane-1,3-dicarbohydrazide;
Bis [N′-(2-napthyl)methylene)]adamantane-1,3-dicarbohydrazide;
Bis [N′-(5-nitro, furan-2-yl)methylene)]adamantane-1,3-dicarbohydrazide;
Bis [N′-(3-nitro, 4-hydroxy, 5-methoxy) benzylidene]adamantane-1,3-dicarbohydrazide;
Bis[N′-(4-methyl)benzylidene]adamantane-1,3-dicarbohydrazide;
Bis[N′-(4-hydroxy)benzylidene]adamantane-1,3-dicarbohydrazide;
Bis[N′-(1-(4-hydroxyphenyl)ethylidene)]adamantane-1,3-dicarbohydrazide;
Bis[N′-(3-bromo,4hydroxy,5-methoxy)benzylidene]adamantane-1,3-dicarbohydrazide;
Bis[N′-thiophene-2-yl)methylene]adamantane-1,3-dicarbohydrazide;
Bis[N′-(4-ethoxy)benzylidene]adamantane-1,3-dicarbohydrazide;
Bis[N′-(3-nitro,4-chloro)benzylidene]adamantane-1,3-dicarbohydrazide;
Bis[N′-(4-bromo)benzylidene]adamantane-1,3-dicarbohydrazide;
Bis[N′-(4-methoxy)benzylidene]adamantane-1,3-dicarbohydrazide;
Bis[N′-(1-(3-nitro,4-chloro)ethylidene)]adamantane-1,3-dicarbohydrazide;
   a stereoisomer, a tautomer, a pharmaceutically acceptable salt or a pharmaceutically acceptable solvate thereof.

In an aspect, the present disclosure provides a pharmaceutical composition comprising a compound of Formula I, a stereoisomer, a tautomer, a pharmaceutically acceptable salt or a pharmaceutically acceptable solvate thereof and a pharmaceutically acceptable excipient.

In an aspect, the present disclosure provides a process of synthesis of compound of Formula I, a stereoisomer, a tautomer, a pharmaceutically acceptable salt or a pharmaceutically acceptable solvate thereof comprising the steps of:
(a) reacting a dimethyl-1,3-adamantane-di-carboxylate compound of Formula IV with hydrazine hydrate in the presence of an alcoholic solution to give a dihydrazide compound of Formula III; and

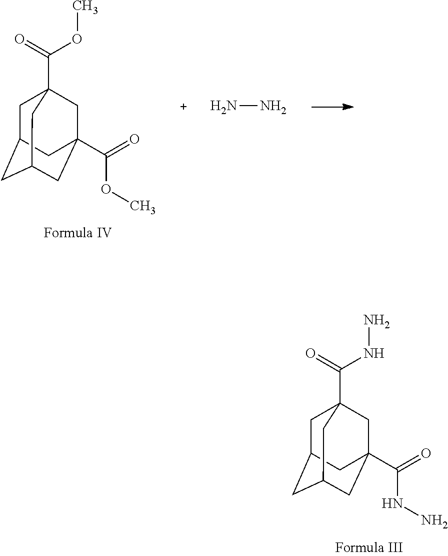

(b) refluxing the compound of Formula III with a compound of Formula II in the presence of a solvent to give the compound of Formula I

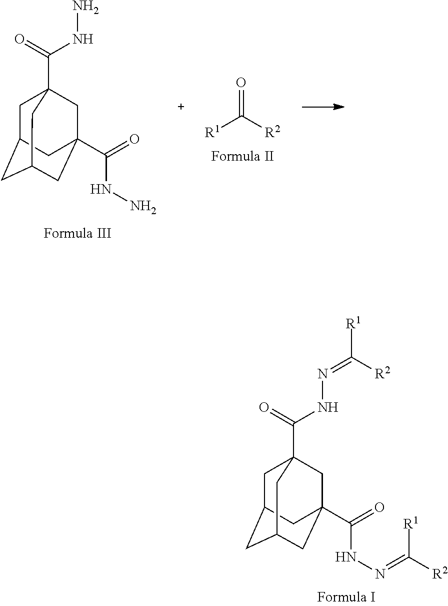

wherein the terms R¹ and R² have the meanings as defined above.

Other aspects of the invention will be set forth in the description which follows, and in part will be apparent from the description, or may be learnt by the practice of the invention.

### DETAILED DESCRIPTION OF THE INVENTION

The following is a detailed description of embodiments of the disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In some embodiments, numbers have been used for quantifying weights, percentages, ratios, and so forth, to describe and claim certain embodiments of the invention and are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Various terms as used herein are shown below. To the extent a term used in a claim is not defined below, it should be given the broadest definition persons in the pertinent art have given that term as reflected in printed publications and issued patents at the time of filing.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context requires otherwise, throughout the specification which follow, the word “comprise” and variations thereof, such as, “comprises” and “comprising” are to be construed in an open, inclusive sense that is as “including, but not limited to.”

The recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified.

The description that follows, and the embodiments described therein, is provided by way of illustration of an example, or examples, of particular embodiments of the principles and aspects of the present disclosure. These examples are provided for the purposes of explanation, and not of limitation, of those principles and of the disclosure.

It should also be appreciated that the present disclosure can be implemented in numerous ways, including as a system, a method or a device. In this specification, these implementations, or any other form that the invention may take, may be referred to as processes. In general, the order of the steps of the disclosed processes may be altered within the scope of the invention.

The headings and abstract of the invention provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

The term “or”, as used herein, is generally employed in its sense including “and/or” unless the content clearly dictates otherwise.

The term, “(C₁₋₆)alkyl”, as used herein, refers to saturated aliphatic groups, including straight or branched-chain alkyl groups having six or fewer carbon atoms in its backbone, for instance, C₁-C₆ for straight chain and C₃-C₆ for branched chain. As used herein, (C₁₋₆)alkyl refers to an alkyl group having from 1 to 6 carbon atoms. Representative examples of alkyl include, but are not limited to, methyl, ethyl, n-propyl, n-butyl, n-pentyl, n-hexyl, isopropyl, sec-butyl, isobutyl, tert-butyl, isopentyl, 2-methylbutyl and 3-methylbutyl.

Furthermore, unless stated otherwise, the alkyl group can be unsubstituted or substituted with one or more substituents, for example, from one to four substituents, independently selected from the group consisting of halogen, hydroxy, cyano, nitro and amino. Examples of substituted alkyl include, but are not limited to hydroxymethyl, 2-chlorobutyl, trifluoromethyl and aminoethyl.

The term, “(C₁₋₆)alkoxy” refers to a (C₁₋₆)alkyl having an oxygen attached thereto.

Representative examples of alkoxy groups include, but are not limited to, methoxy, ethoxy, propoxy, isopropoxy, n-butoxy, isobutoxy and tert-butoxy. Furthermore, unless stated otherwise, the alkoxy groups can be unsubstituted or substituted with one or more groups. A substituted alkoxy refers to a (C₁₋₆)alkoxy substituted with one or more groups, particularly one to four groups independently selected from the groups indicated above as the substituents for the alkyl group.

The term “(C₆₋₁₀)aryl” or “aryl” as used herein refers to monocyclic or bicyclic hydrocarbon groups having 6 to 10 ring carbon atoms, wherein at least one carbocyclic ring is having a n electron system. Examples of (C₆-C₁₀) aryl ring systems include, but are not limited to, phenyl and naphthyl. Unless indicated otherwise, aryl group can be unsubstituted or substituted with one or more substituents, for example 1-4 substituents independently selected from the group consisting of halogen, (C₁₋₆)alkyl, hydroxy, cyano, nitro, —COOH, amino and (C₁₋₆) alkoxy.

The term, (C₄₋₁₀)heterocyclyl, as used herein refers to a 4- to 10-membered, saturated, partially unsaturated or unsaturated monocyclic or bicyclic ring system containing 1 to 4 heteroatoms independently selected from the group consisting of oxygen, nitrogen and sulfur.

Saturated heterocyclic ring systems do not contain any double bond, whereas partially unsaturated heterocyclic ring systems contains at least one double bond, and unsaturated heterocyclic ring systems form an aromatic system containing heteroatom(s). The oxidized form of the ring nitrogen and sulfur atom contained in the heterocyclyl to provide the corresponding N-oxide, S-oxide or S,S-dioxide is also encompassed in the scope of the present invention. Representative examples of heterocyclyls include, but are not limited to, tetrahydrofuran, tetrahydrothiophene, pyrrolidine, dihydropyran, tetrahydropyran, thio-dihydropyran, thio-tetrahydropyran, piperidine, piperazine, morpholine, 1,3-oxazinane, 1,3-thiazinane, 4,5,6-tetrahydropyrimidine, 2,3-dihydrofuran, dihydrothiene, dihydropyridine, tetrahydropyridine, isoxazolidine, pyrazolidine, furan, pyrrole, thiophene, imidazole, oxazole, thiazole, triazole, tetrazole, benzofuran, indole, benzoxazole, benzothiazole, isoxazole, triazine, purine, pyridine, pyrazine, quinoline, isoquinoline, phenazine, oxadiazole, pteridine, pyridazine, quinazoline, pyrimidine, isothiazole, benzopyrazine and tetrazole. Unless stated otherwise, (C₄₋₁₀)heterocyclyl can be unsubstituted or substituted with one or more substituents, for example, substituents independently selected from the group consisting of oxo, halogen, hydroxy, cyano, nitro, amine, (C₁₋₆)alkyl and COOH.

The term, “halogen” as used herein refers to chlorine, fluorine, bromine or iodine atom.

The term, “therapeutically effective amount” or “effective amount” as used herein refers to an amount of a compound of Formula I or a pharmaceutically acceptable salt thereof or a composition comprising a compound of Formula I or a salt thereof, effective in producing the desired therapeutic response in a particular patient (subject) suffering from a disease or disorder.

The term “pharmaceutically acceptable excipient(s)” as used herein refers to a diluent, binder, disintegrant, glidant, lubricant, coating material or the like, which is non-toxic, and inert, which does not have undesirable effects on a subject to whom it is administered and is suitable for delivering a therapeutically active agent to the target site without affecting the therapeutic activity of the said agent.

The term, “subject” as used herein refers to an animal, preferably a mammal, and most preferably a human. The term “mammal” used herein refers to warm-blooded vertebrate animals of the class ‘mammalia’, including humans, characterized by a covering of hair on the skin and, in the female, milk-producing mammary glands for nourishing the young, the term mammal includes animals such as cat, dog, rabbit, bear, fox, wolf, monkey, deer, mouse, pig and human.

The terms, “treatment”, “treat” and “therapy” and the like as used herein refer to alleviate, slow the progression, attenuation, prophylaxis or as such treat the existing diseases or condition (e.g. bacterial infection or fungal infection). Treatment also includes treating, preventing development of, or alleviating to some extent, one or more of the symptoms of the diseases or condition.

Aspects of the present disclosure provide dihydrazide-dihydrazone compounds with adamantyl moiety possessing antimicrobial activity.

In an embodiment, the present disclosure provides a compound of Formula I, a stereoisomer, a tautomer, a pharmaceutically acceptable salt or a pharmaceutically acceptable solvate thereof,

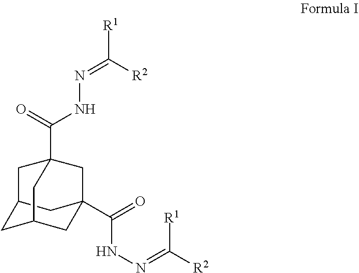

wherein R¹ is an unsubstituted or substituted C₆₋₁₀ aryl or an unsubstituted or substituted C₄₋₁₀ heterocyclyl;
R² is selected from H, C₁₋₆alkyl, C₁₋₆alkoxy, halogen, nitro, —COOH or combinations thereof; and wherein the substituents are selected from one or more of halogen, nitro, C₁₋₆alkyl, C₁₋₆alkoxy, —COOH, hydroxy, cyano, or NH₂.

In an embodiment, R¹ is unsubstituted or substituted phenyl or napthyl; wherein the substituents are selected from one or more of halogen, nitro, hydroxy, C₁₋₆alkoxy, or C₁₋₆alkyl.

In an embodiment, R¹ is unsubstituted or substituted C₄₋₁₀ heterocyclyl, preferably furanyl or thienyl; wherein the substituents are selected from one or more of nitro, hydroxy, C₁₋₆alkoxy, or C₁₋₆alkyl.

In an embodiment, the C₁₋₆alkoxy is methoxy, or ethoxy. In an embodiment, the C₁₋₆ alkyl is methyl.

In an embodiment, R² is selected from H or C₁₋₆alkyl. In an embodiment, the C₁₋₆alkyl is methyl.

In an embodiment, the compound of Formula I may be selected from:
Bis [N′-(4-nitro)benzylidene]adamantane-1,3-dicarbohydrazide;
Bis [N′-(3-methoxy, 4-hydroxy)benzylidene]adamantane-1,3-dicarbohydrazide;
Bis [N′-(1-(2-hydroxy, 5-methyl phenyl)ethylidene)]adamantane-1,3-dicarbohydrazide;
Bis [N′-(3,4-dimethoxy)benzylidene)]adamantane-1,3-dicarbohydrazide;
Bis [N′-(1-(4-ethoxy)ethylidene)]adamantane-1,3-dicarbohydrazide;
Bis [N′-(2-napthyl)methylene)]adamantane-1,3-dicarbohydrazide;
Bis [N′-(5-nitro, furan-2-yl)methylene)]adamantane-1,3-dicarbohydrazide;
Bis [N′-(3-nitro, 4-hydroxy, 5-methoxy) benzylidene]adamantane-1,3-dicarbohydrazide;
Bis[N′-(4-methyl)benzylidene]adamantane-1,3-dicarbohydrazide;
Bis[N′-(4-hydroxy)benzylidene]adamantane-1,3-dicarbohydrazide;
Bis[N′-(1-(4-hydroxyphenyl)ethylidene)]adamantane-1,3-dicarbohydrazide;
Bis[N′-(3-bromo,4hydroxy,5-methoxy)benzylidene]adamantane-1,3-dicarbohydrazide;
Bis[N′-thiophene-2-yl)methylene]adamantane-1,3-dicarbohydrazide;
Bis[N′-(4-ethoxy)benzylidene]adamantane-1,3-dicarbohydrazide;
Bis[N′-(3-nitro,4-chloro)benzylidene]adamantane-1,3-dicarbohydrazide;
Bis[N′-(4-bromo)benzylidene]adamantane-1,3-dicarbohydrazide;
Bis[N′-(4-methoxy)benzylidene]adamantane-1,3-dicarbohydrazide;
Bis[N′-(1-(3-nitro,4-chloro)ethylidene)]adamantane-1,3-dicarbohydrazide;
   a stereoisomer, a tautomer, a pharmaceutically acceptable salt or a pharmaceutically acceptable solvate thereof.

In another embodiment, the compound of Formula I can be converted into a pharmaceutically acceptable salt. The pharmaceutically acceptable salts of the compound of Formula I according to the disclosure are prepared in a manner known to one skilled in the art. Pharmaceutically acceptable salts of the compound of the present disclosure include but are not limited to, an acid salt of a compound of the present disclosure containing an amine or other basic group can be obtained by reacting the compound with a suitable organic or inorganic acid, resulting in pharmaceutically acceptable anionic salt forms. Examples of anionic salts include the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate, and triethiodide salts.

In yet another embodiment, the pharmaceutically acceptable salts of the compound of the present disclosure containing acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine.

In an embodiment, the compounds of the present disclosure are antimicrobials. In preferred embodiments, the compounds of the present disclosure have antibacterial activity, antifungal activity, or combination thereof.

In particular embodiments, compounds of the present disclosure are active against pathogens selected from Staphylococcus sp., Candida sp., Escherichia sp., Shigella, Streptococcus, Pseudomonas sp., Klebsiella sp., Enterobacter sp., Bacillus sp., Enterococcus sp., Aspergillus sp., or their combinations.

In an embodiment, the present disclosure provides a pharmaceutical composition comprising a compound of Formula I, a stereoisomer, a tautomer, a pharmaceutically acceptable salt or a pharmaceutically acceptable solvate thereof and a pharmaceutically acceptable excipient.

The present disclosure also relates to a process for the production of the pharmaceutical composition, which includes bringing a compound of Formula I, into a suitable administration form using a pharmaceutically acceptable excipient or a carrier and, if appropriate, further suitable pharmaceutically acceptable carriers, additives or auxiliaries. The pharmaceutical compositions containing the compound of Formula I according to the disclosure are prepared in a manner known to one skilled in the art.

In an embodiment, the pharmaceutical compositions can be administered orally, for example in the form of pills, tablets, coated tablets, capsules, granules or elixirs. Administration, however, can also be carried out rectally, for example in the form of suppositories, or parenterally, for example intravenously, intramuscularly or subcutaneously, in the form of injectable sterile solutions or suspensions, or topically, for example in the form of ointments or creams or transdermally, in the form of patches, or in other ways, for example in the form of aerosols or nasal sprays.

For the production of oral dosage forms of the compound of Formula I such as the pills, tablets, coated tablets and hard gelatin capsules, it is possible to use, for example, lactose, corn starch or compounds thereof, gum arabica, magnesia or glucose, etc. Pharmaceutically acceptable excipients that can be used for soft gelatin capsules and suppositories are, for example, fats, waxes, natural or hardened oils, etc. Suitable pharmaceutically acceptable excipients for the production of solutions, for example injection solutions, or of emulsions or syrups are, for example, water, physiological sodium chloride solution or alcohols, for example, ethanol, propanol or glycerol, sugar solutions, such as glucose solutions or mannitol solutions, or a mixture of the said solvents.

In another embodiment, the pharmaceutical compositions normally contain about 1% to 99%, for example, about 5% to 70%, or from about 10% to about 30% by weight of the compound of Formula I or its pharmaceutically acceptable salt. The amount of the compound of Formula I or its pharmaceutically acceptable salt in the pharmaceutical compositions normally is from about 5 to 500 mg or may be lower than or higher than the lower and the upper limit respectively. The dose of the compound of Formula I, which is to be administered, can cover a wide range depending on the type of disease or disorder to be treated. The dose to be administered daily is to be selected to suit the desired effect. A suitable dosage is about 0.01 to 100 mg/kg of the compound of Formula I or its pharmaceutically acceptable salt depending on the body weight of the recipient (subject) per day, for example, about 0.1 to 50 mg/kg/day of a compound of Formula I or a pharmaceutically acceptable salt of the compound. If required, higher or lower daily doses can also be administered.

The selected dosage level will depend upon a variety of factors including the activity of a compound of the present disclosure, or its salt employed, the route of administration, the time of administration, the rate of excretion of the particular compound being administered, the duration of the treatment, other concurrently administered drugs, compounds and/or materials, the age, sex, weight, condition, general health and prior medical history of the patient (subject) being treated, and like factors well known in the medical arts.

In addition to the compound of Formula I or its pharmaceutically acceptable salt and the pharmaceutically acceptable carrier substances, the pharmaceutical compositions of the present invention can contain excipients such as, for example, fillers, antioxidants, dispersants, emulsifiers, defoamers, flavors, preservatives, solubilizers or colorants. Furthermore, in addition to a compound of Formula I or its pharmaceutically acceptable salt, the pharmaceutical compositions can also contain one or more other therapeutically or prophylactically active agents.

The present disclosure also encompasses within its scope the use of a compound of Formula I in a medicament, optionally with other therapeutically active agents. In an embodiment, the combination of compound of present invention with another therapeutic agent or treatment includes co-administration of a compound of Formula I with the other therapeutic agent or treatment as either a single combination dosage form or as multiple, separate dosage forms, administration of the compound of the present invention first, followed by the other therapeutic agent or treatment and administration of the other therapeutic agent or treatment first, followed by the compound of present disclosure. Further therapeutic agents are administered either simultaneously or sequentially.

In another embodiment of the present disclosure, the other therapeutic agent may be any agent that is known in the art to treat, prevent, or reduce the symptoms of a disease or disorder. The selection of other therapeutic agent(s) is based upon the particular disease or disorder being treated. Such choice is within the knowledge of a treating physician. Furthermore, the additional therapeutic agent may be any agent when administered in combination with the administration of a compound of the present invention provides benefit to the subject in need thereof.

In an embodiment, the present disclosure provides a process of synthesis of compound of Formula I via condensation of dihydrazides with aldehydes or ketones.

In an embodiment, the process of synthesis of compound of Formula I, a stereoisomer, a tautomer, a pharmaceutically acceptable salt or a pharmaceutically acceptable solvate thereof comprises the steps of:
(a) reacting a dimethyl-1,3-adamantane-di-carboxylate compound of Formula IV with hydrazine hydrate in the presence of an alcoholic solution to give a dihydrazide compound of Formula III; and

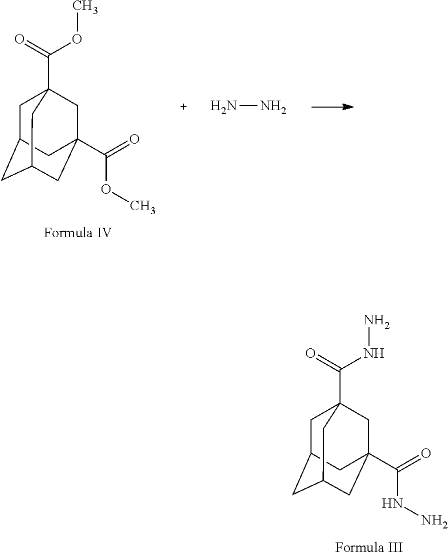

(b) refluxing the compound of Formula III with a compound of Formula II in the presence of a solvent to give the compound of Formula I

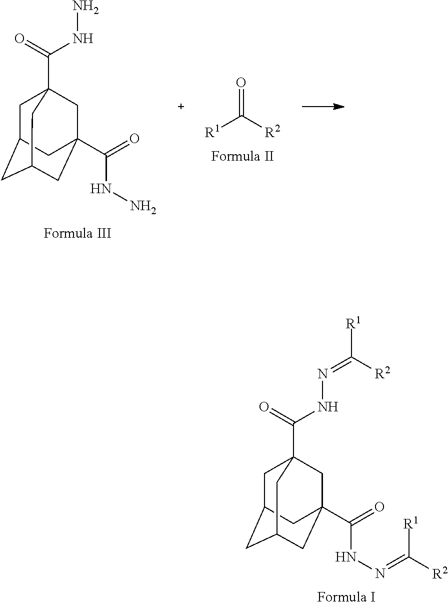

wherein the terms R¹ and R² have the meanings as defined above.

In an embodiment, the alcoholic solution may be ethanolic or methanolic solution. Alternatively any other well-known alcoholic solution may be employed. The process of step (a) is performed under low temperatures, preferably 0-5° C.

In an embodiment, the solvent may be selected from any solvent conventionally known in the art for condensation, including but not limited to, methanol, ethanol, or combinations thereof.

In an embodiment, the compounds of Formula III and Formula II were reacted in a molar ratio range of about 1:1 to about 1:5, preferably the ratio is 1:2.

In an embodiment, the compound of Formula IV may be synthesized by esterification by refluxing a dicarboxylic compound of Formula V with methanol in sulphuric acid

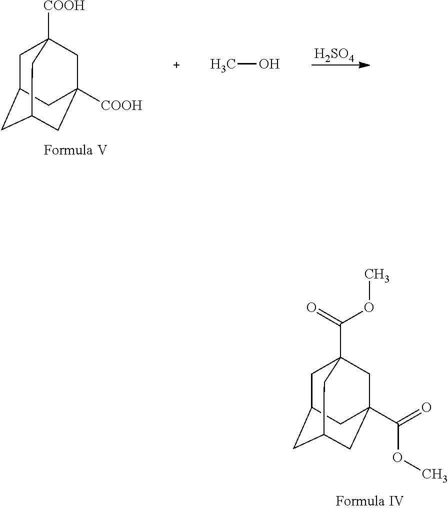

In a preferred embodiment, the esterification may be catalyzed by 98% sulfuric acid.

The process of synthesis of the compounds is simple, facile and cost effective. The process is also industrially scalable.

In an embodiment, the present disclosure provides a method of treatment, amelioration or prophylaxis of an antimicrobial infection by administering a therapeutically effective amount of a compound of Formula I, a stereoisomer, a tautomer, a pharmaceutically acceptable salt or a pharmaceutically acceptable solvate thereof.

In an embodiment, the present disclosure provides a method of inhibiting the growth of a microbe by adding an effective amount of a compound of Formula I, a stereoisomer, a tautomer, a pharmaceutically acceptable salt or a pharmaceutically acceptable solvate thereof.

While the foregoing describes various embodiments of the disclosure, other and further embodiments of the disclosure may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### EXAMPLES

The present invention is further explained in the form of following examples. However, it is to be understood that the following examples are merely illustrative and are not to be taken as limitations upon the scope of the invention.

MATERIALS AND METHODS: All reagents were received from commercial suppliers. Melting points (M.P.) of synthesized compounds were confirmed by open capillary tube method. All compounds were tested for FTIR spectra and elemental analysis. ¹H NMR spectra were recorded using 600.1723046 [MHz] NMR spectrophotometer. The chemical shifts were expressed in ppm and constants were given in Hz using tetramethylsilane as the internal standard. For recording NMR spectra, DMSO-D6 was used as a solvent. Reference peak was at 2.5 ppm.

### Example 1

### Synthesis of the Compounds

The compounds of the present disclosure were synthesized using the General Scheme I given below. Dimethyl-1,3-adamantane-di-carboxylate (2) was used as the key intermediate. It was prepared by esterification of 1,3-adamantane-di-carboxylic acid (1) and methanol under catalysis of 98% H₂SO₄ to yield corresponding dimethyl ester (2). Then, ester (2) was reacted with 80% hydrazine hydrate to yield corresponding adamantane dihydrazide (3). Subsequently, compound (3) was condensed with aromatic aldehydes or ketones (4) to yield the corresponding dihydrazide-dihydrazones Compounds (5).

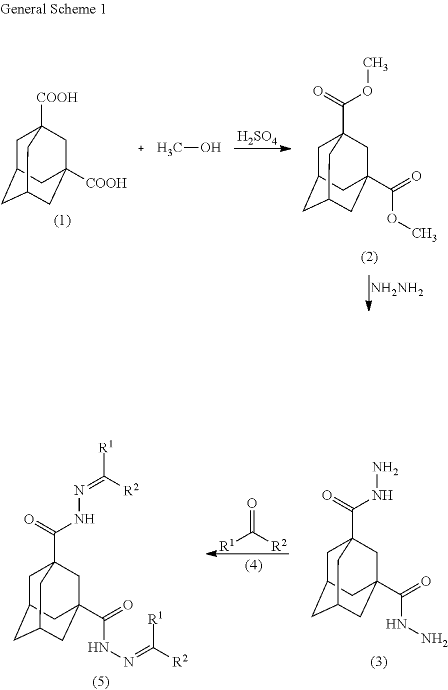

### (a) Synthesis of dimethyl-1,3-adamantane dicarboxylate (2) from adamantane-1,3-dicarboxylic acid (1)

A mixture of 0.02 mol of adamantane-1,3-dicarboxylic acid and 15 ml of methanol in presence of 98% H₂SO₄ as catalyst was refluxed. The mixture was cooled and washed with water. This mixture was successively washed with sodium hydrogen carbonate (15% NaHCO₃ aqueous solution) till pH 7 followed by washing with water again. The organic phase was dried over anhydrous sodium sulfate (Na₂SO₄), evaporated and crystallized.

### (b) Synthesis of Adamantane-1,3-dicarbohydrazide(3) from dimethyl-1,3-adamantane dicarboxylate (2)

To a methanolic solution (excess methanol) of dimethyl-1,3-adamantane-di-carboxylate(1.00924 g, 0.004 mol), (0.08 mol) 80% hydrazine hydrate was added dropwise with constant stirring. The reaction was carried out at 0 degree Celsius. White colored Adamantane-1,3-dicarbohydrazide (3) was obtained and was recrystallized in excess methanol till crystallization. Excess amount of methanol is removed. The compound was confirmed from melting point determination.

### (c) Synthesis of dihydrazone Compounds (5) from Adamantane-1,3-dicarbohydrazide(3)

The adamantane-1,3-dicarbohydrazide on condensation with different aldehydes or ketones(4)(in mol ratio 1:2) gives the corresponding dihydrazide-dihydrazone compounds. The dicarbohydrazide was refluxed at around 85-110° C. in the presence of excess methanol. The reaction was monitored for completion by TLC.

Dihydrazide-dihydrazone Compounds (5a-5r) synthesized by the scheme above have been provided in the Table 1 below. All synthesized compounds were characterized by FTIR, ¹H-NMR, and elemental analysis.

TABLE 1
Structure and characterization data for the synthesized compounds

| Structure | Characterization Data |
|---|---|
| 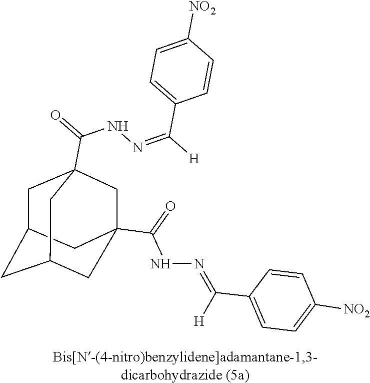 [SMILES: O=C(N/N=C\c1ccc([N+](=O)[O-])cc1)C12CC3CC(C1)CC(C(=O)N/N=C/c1ccc([N+](=O)[O-])cc1)(C3)C2] | M.P.: 181° C., Yield 77.64%, light yellow, Mol. Wt. 518.52124 ¹H-NMR (600 MHz, DMSO-d6, δ ppm): 10.2-8.8 (1H, s, NH—N); 8.15-8.44 (Ar—H); 3.4-3.6 (═CH); 1.62-2.07 (Adamantane-H) IR (ν cm⁻¹): 1104 (N—N); 1247 (C—N for NO2); 1286(C—O); 1345 (C—N); 1524 (NO2); 1599 (C═N); 1709 (C═O); 2850 (N—H); 2921-2996 (C—H); 3106 (H—C═) Analysis: for C₂₆H₂₆N₆O₆ found (calculated): C, 57.40 (60.22); H, 3.87 (5.05); N, 9.43 (16.21)% |
| 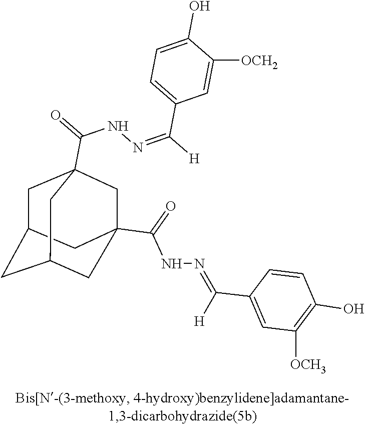 | M.P.: 158° C., Yield 84.43 %, Light yellow to light brown, Mol. Wt. 520.57688 ¹H-NMR (600 MHz, DMSO-d6, δ ppm): 9.7 (1H, s, NH—N); 8.56 (—OH attached to benzene); 3.4-3.6 (═CH); 3.85 (—OCH3); 6.86-7.39 (Ar—H); 1.62-2.07 (Adamantane-H) IR (ν cm⁻¹): 1097 (N—N); 1214-1273 (C—N); 1513 (C═C benzene); 1598 (C═N); 1727 (C═O); 2857 (N—H); 2920-3069 (C—H); 3190 (—OH); 3410 (H—C═) Analysis: for C₂₈H₃₂N₄O₆ found (calculated): C, 64.69 (64.60); H, 6.15 (6.20); N, 3.93 (10.76)% |
| 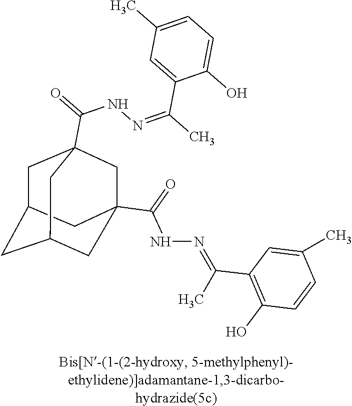 [SMILES: C/C(=N/NC(=O)C12CC3CC(C1)CC(C(=O)N/N=C(\C)c1cc(C)ccc1O)(C3)C2)c1cc(C)ccc1O] | M.P.: 221° C., Yield 72 %, Shiny dark yellow, Mol. Wt. 516.63124 ¹H-NMR (600 MHz, DMSO-d6, δ ppm): 12.75 (—OH attached to benzene ring) 7.58(1H, s, NH—N); 6.86-7.3 (Ar—H); 3.56 (═CH); 1.62-2.00 (Adamantane-H) IR (ν cm⁻¹): 1039 (N—N); 1233 (C—O); 1290 (C—N); 1563-1492 (C═C benzene); 1614 (C═N); 1744 (C═O); 2920 (N—H); 2993 (C—H); 3434 (H—C═); 3752 (—OH). Analysis: for C₃₀H₃₆N₄O₄ found (calculated): C, 71.44 (69.74); H, 6.27 (7.02); N, 9.08 (10.84)% |
| 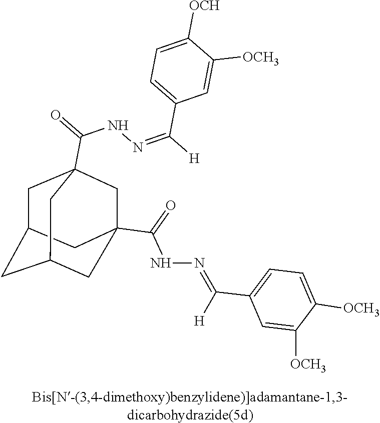 | M.P.: 215° C., Yield 71.90 %, Light yellow, Mol. Wt. 548.63004 ¹H-NMR (600 MHz, DMSO-d6, δ ppm): 8.6(1H, s, NH—N); 7.06-7.48 (Ar—H); 3.37 (═CH); 1.62-2.00 (Adamantane-H). IR (ν cm⁻¹): 1016 (N—N); 1157 (C—O); 1259 (C—N); 1508-1464 (C═C benzene); 1623 (C═N); 1729 (C═O); 2839 (N—H); 2929 (C—H); 2961 (H—C═). Analysis: for C₃₀H₃₆N₄O₆ found (calculated): C, 65.86 (65.68); H, 5.91 (6.61); N, 8.39 (10.21)% |
| 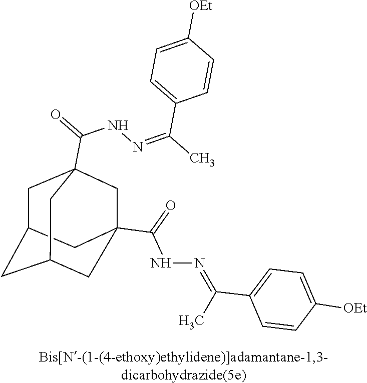 [SMILES: CCOc1ccc(/C(C)=N\NC(=O)C23CC4CC(C2)CC(C(=O)N/N=C(\C)c2ccc(OCC)cc2)(C4)C3)cc1] | M.P.: 185° C., Yield 73.26%, Yellow, Mol. Wt. 516.63124 ¹H-NMR (600 MHz, DMSO-d6, δ ppm): 7.91-7.93(1H, s, NH—N); 7.1-7.86 (Ar—H); 4.06-4.13 (—OC2H5); 3.5 (═CH); 1.62-2.00 (Adamantane-H). IR (ν cm⁻¹): 1043 (N—N); 1172 (C—O); 1298 (C—N); 1505 (C═C benzene); 1598 (C═N); 1681 (C═O); 2932 (N—H); 2979 (C—H); 3447 (H—C═). Analysis: for C₃₂H₄₀N₄O₄ found (calculated): C, 73.94 (69.74); H, 7.42 (7.02); N, 8.21 (10.84)% |
| 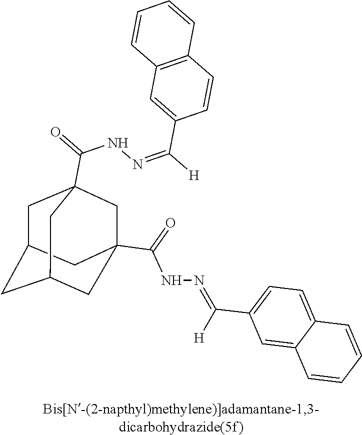 [SMILES: O=C(N/N=C\c1ccc2ccccc2c1)C12CC3CC(C1)CC(C(=O)N/N=C/c1ccc4ccccc4c1)(C3)C2] | M.P.: 241° C., Yield 18.91 %, Light yellow, Mol. Wt. 528.64348 ¹H-NMR (600 MHz, DMSO-d6, δ ppm): 8.92(1H, s, NH—N); 7.6-8.38 (napthalene—H); 3.4 (═CH); 1.62-2.00 (Adamantane-H). IR (ν cm⁻¹): 1016 (N—N); 1172 (C—N); 1502 (C═C benzene); 1616 (C═N);1692 (C═O); 2800 (N—H); 3054 (C—H); 3434 (H—C═). Analysis: for C₃₄H₃₂N₄O₂ found (calculated): C, 85.28 (77.25); H, 4.54 (6.10); N, 8.71 (10.60)% |
| 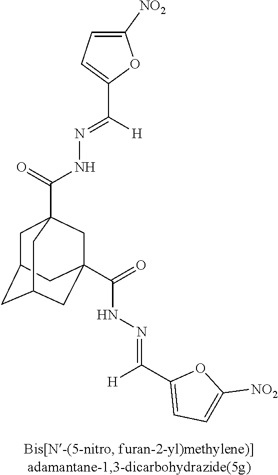 [SMILES: O=C(N/N=C/c1ccc([N+](=O)[O-])o1)C12CC3CC(C1)CC(C(=O)N/N=C/c1ccc([N+](=O)[O-])o1)(C3)C2] | M.P.: 186° C., Yield 84.41 %, Dark brown, Mol. Wt. 498.44548 ¹H-NMR (600 MHz, DMSO-d6, δ ppm): 6.9-7.6(1H, s, NH—N); 3.5-3.6 (═CH broad peak); 1.62-2.00 (Adamantane-H). IR (ν cm⁻¹): 1026 (N—N); 1220-1280 (C—N); 1405 (NO2); 1610 (C═N); 1728 (C═O); 2863 (N—H); 2920 (C—H); 3430 (H—C═). Analysis: for C₂₂H₂₂N₆O₈ found (calculated): C, 58.32 (53.01); H, 6.76 (4.45); N, 3.97 (16.86)% |
| 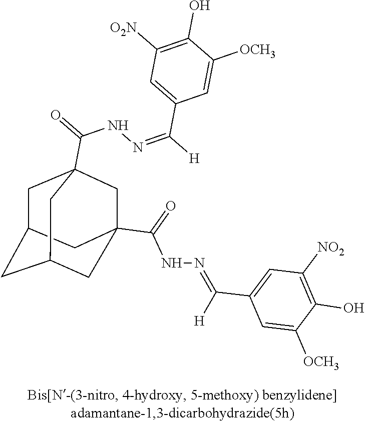 [SMILES: COc1cc(/C=N\NC(=O)C23CC4CC(C2)CC(C(=O)N/N=C/c2cc(OC)c(O)c([N+](=O)[O-])c2)(C4)C3)cc([N+](=O)[O-])c1O] | M.P.: 180° C., Yield 72.03 %, Brown, Mol. Wt. 609.798 ¹H-NMR (600 MHz, DMSO- d6, δ ppm): 9.9 (—OH attached to benzene); 9.8(1H, s, NH—N); 7.62-8.1 (Ar—H); 3.4 (═CH); 1.62-2.00 (Adamantane-H). IR (ν cm⁻¹): 1049 (N—N); 1111 (C—O); 1235-1273 (C—N); 1406 (NO2); 1577 (N—O); 1614 (C═N); 1689 (C═O); 2871 (N—H); 2992 (C—H); 3216 (H—C═). Analysis: for C₂₈H₃₀N₆O₁₀ found (calculated): C, 49.11 (55.08); H, 3.34 (4.95); N, 6.87 (13.76)% |
| 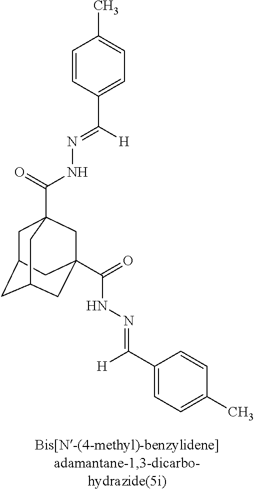 [SMILES: Cc1ccc(/C=N/NC(=O)C23CC4CC(C2)CC(C(=O)N/N=C/c2ccc(C)cc2)(C4)C3)cc1] | M.P.: 53° C., Yield 60.00%, light yellow, Mol. Wt. 456.57928 ¹H-NMR (600 MHz, DMSO-d6, δ ppm): 9.9-8.65(1H, s, NH—N); 7-8.5 (A—H); 3.56 (═CH); 2.7 (—CH₃); 1.62-2.00 (Adamantane-H) IR (ν cm⁻¹): 1091 (N—N); 1245-1281 (C—N); 1511-1513 (C═C benzene); 1610 (C═N); 1726 (C═O); 2863 (N—H); 2921- 2993 (C—H); 3430 (H—C═) Analysis: for C₂₈H₃₂N₄O₂ found (calculated): C, 73.63 (73.66); H, 7.06 (7.06); N, 12.25 (12.27)% |
| 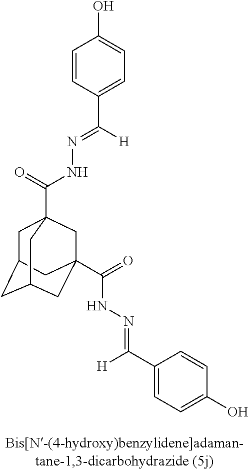 [SMILES: O=C(N/N=C/c1ccc(O)cc1)C12CC3CC(C1)CC(C(=O)N/N=C/c1ccc(O)cc1)(C3)C2] | M.P.: 160° C., Yield 80.33 %, Light brown, Mol.Wt. 460.52492 ¹H-NMR (600 MHz, DMSO-d6, δ ppm): 9.8(1H, s, NH—N); 8.55 (—OH attached to benzene); 3.35-3.6 (═CH); 6.84-7.77 (Ar—H); 1.62- 2.08 (Adamantane-H) IR (ν cm⁻¹): 1095 (N—N); 1285 (C—N); 1603 (C═N); 1727 (C═O); 2859 (N—H); 2946 (H—C═); 3168 (OH) Analysis: for C₂₆H₂₈N₄O₄ found (calculated): C, 67.79 (67.81); H, 6.14 (6.13); N, 12.15 (12.17)% |
| 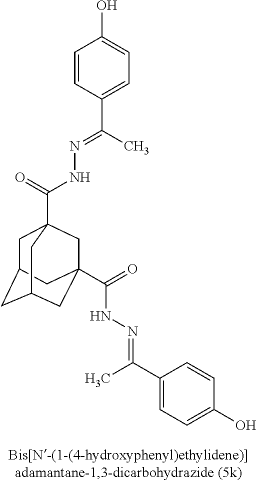 [SMILES: C/C(=N\NC(=O)C12CC3CC(C1)CC(C(=O)N/N=C(\C)c1ccc(O)cc1)(C3)C2)c1ccc(O)cc1] | M.P.: 184° C., Yield 83.96%, Light Brown; Mol. Wt. 488.57808 ¹H-NMR (600 MHz, DMSO-d6, δ ppm): 10.34(1H, s, NH—N); 8.3 (—OH attached to benzene); 3.35-3.6 (—CH₃); 6.86-7.82 (Ar—H); 1.62- 2.08(Adamantane-H) IR (ν cm⁻¹): 1095.06 (N—N); 1280 (C—N); 1511 (C═C benzene); 1604 (C═N); 1727 (C═O); 2859 (N—H); 2919-2994 (C—H); 3310 (—OH); 3429 (H—C═) Analysis: for C₂₈H₃₂N₄O₄ found (calculated): C, 8.80 (68.83); H, 6.64 (6.60); N, 11.45 (11.47)% |
| 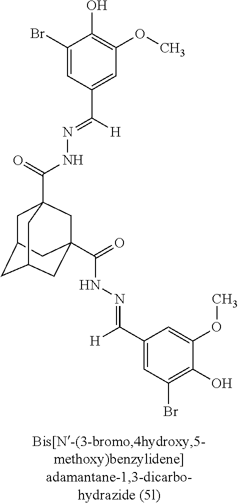 [SMILES: COc1cc(/C=N/NC(=O)C23CC4CC(C2)CC(C(=O)N/N=C/c2cc(Br)c(O)c(OC)c2)(C4)C3)cc(Br)c1O] | M.p.: 285° C., Yield 77.47 %, Dark yellow, Mol. Wt. 678.369 ¹H-NMR (600 MHz, DMSO-d6, δ ppm): 8.7(1H, s, NH—N); 7.96-7.73 (Ar—H); 4.0 (—OH attached to benzene); ); 3.955 (—OCH₃); 3.4 (═CH); 1.62-2.08 (Adamantane-H) IR (ν cm⁻¹): 611-644 (C—Br stretching); 1060 (N—N); 1271 (C—N); 1541 (C═C benzene); 1628 (C═N); 1697 (C═O); 2946 (N—H); 3083 (H—C═); 3209 (—OH) Analysis: for C₂₈H₃₀N₄Br₂O₆ found (calculated): C, 49.55 (49.57); H, 4.43 (4.46); N, 8.29 (8.26)% |
| 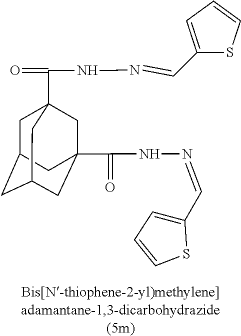 [SMILES: O=C(N/N=C\c1cccs1)C12CC3CC(C1)CC(C(=O)N/N=C/c1cccs1)(C3)C2] | M.P.: 138° C., Yield 55.67 %, Dark brown, Mol. Wt. 440.58156 ¹H-NMR (600 MHz, DMSO-d6, δ ppm): 8.9(1H, s, NH—N); 7.78-8.85 (thiophene ring—H); 3.38-3.59 (═CH); 1.62-2.00 (Adamantane-H) IR (ν cm⁻¹): 698 (C—S); 1027 (C═S); 1091 (N—N); 1281 (C—N); 1609 (C═N); 1725 (C═O); 2864 (N—H); 2921 (C—H); 3433 (H—C═) Analysis: for C₂₂H₂₄N₄O₂S₂ found (calculated): C, 60.01 (59.97); H, 5.52 (5.49); N, 12.70 (12.72)% |
| 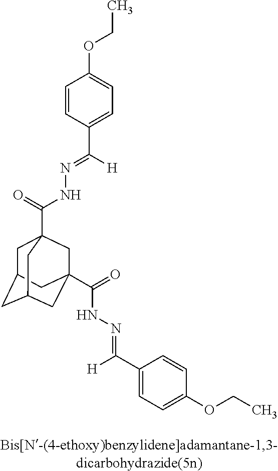 [SMILES: CCOc1ccc(/C=N/NC(=O)C23CC4CC(C2)CC(C(=O)N/N=C/c2ccc(OCC)cc2)(C4)C3)cc1] | M.p.: 58° C., Yield 85.99 %, Yellow, Mol. Wt. 516.63124 ¹H-NMR (600 MHz, DMSO-d6, δ ppm): 9.85-8.65(1H, s, NH—N); 7-7.78 (Ar—H); 4.12-4.14 (—O—C₂H₅); 3.4-3.6 (═CH); 1.6-2.08 (Adamantane-H); 1.33-1.36 (—CH₃) IR (ν cm⁻¹): 1097 (N—N); 1256-1396 (C—N); 1510—1453 (C—C benzene); 1601 (C═N); 1729 (C═O); 2859 (N—H); 2911-3074 (C—H); 3435 (H—C═) Analysis: for C₃₀H₃₆N₄O₄ found (calculated): C, 69.76 (69.74); H, 7.01 (7.02); N, 10.82 (10.84)% |
| 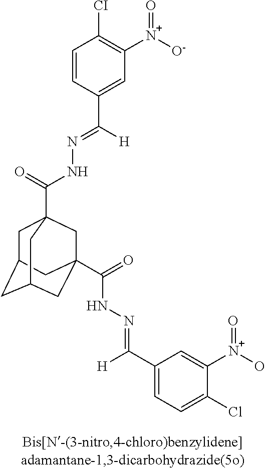 | M.p.: 258° C., Yield 75.84 %, Light yellow, Mol. Wt. 587.41136 ¹H-NMR (600 MHz, DMSO-d6, δ ppm): 8.82(1H, s, NH—N); 7.93-8.5 (Ar—H); 3.37 (═CH); 1.62-2.00 (Adamantane-H) IR (ν cm⁻¹): 838 (C—Cl); 1049 (N—N); 1212-1132 (C—N); 1476 (C—C benzene); 1351 (N—O); 1530 (C—NO₂); 1602 (C═N); 1626 (C═O); 3069 (H—C═) Analysis: for C₂₆H₂₄Cl₂N₆O₆ found (calculated): C, 53.19 (53.16); H, 4.10 (4.12); N, 12.09 (12.07)% |
| 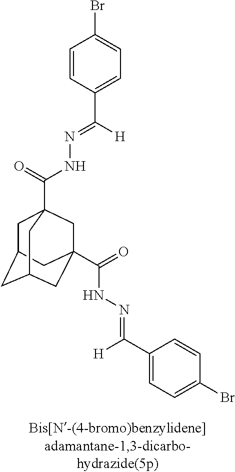 [SMILES: O=C(N/N=C/c1ccc(Br)cc1)C12CC3CC(C1)CC(C(=O)N/N=C/c1ccc(Br)cc1)(C3)C2] | M.p.: 228° C., Yield 41.63 %, Light yellow, Mol. Wt. 586.31824 ¹H-NMR (600 MHz, DMSO-d6, δ ppm): 8.71(1H, s, NH—N); 7.72-7.84 (Ar—H); 3.35 (═CH); 1.62-2.00 (Adamantane-H) IR (ν cm⁻¹): 516-699 (C—Br stretching); 1007 (N—N); 1277 (C—N); 1582-1480 (C═C benzene); 1623 (C═N), 1703 (C—O); 2941 (N—H); 2995-3047 (C—H); 3434 (H—C═) Analysis: for C₂₆H₂₆Br₂N₄O₂ found (calculated): C, 53.24 (53.26); H, 4.45 (4.47); N, 9.53 (9.56)% |
| 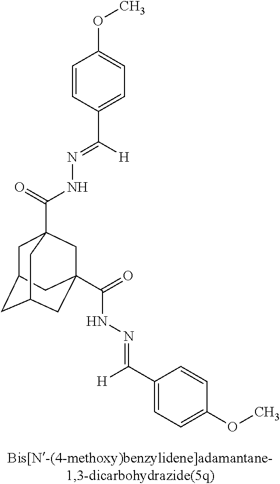 [SMILES: COc1ccc(/C=N/NC(=O)C23CC4CC(C2)CC(C(=O)N/N=C/c2ccc(OC)cc2)(C4)C3)cc1] | M.p.: 176° C., Yield 40.95%, Light yellow, Mol. Wt. 488.57808 ¹H-NMR (600 MHz, DMSO-d6, δ ppm): 8.63(1H, s, NH—N); 7.04-7.82 (Ar—H); 3.4 (═CH); 3.8 (—OCH3); 1.62-2.00 (Adamantane-H) IR (ν cm⁻¹): 1024 (N—N); 1166 (C—O); 1250 (C—N); 1507-1461 (C═C benzene); 1621 (C═N); 1658-1777 (C═O); 2986 (N—H); 2838—2967 (C—H); 3434 (H—C═) Analysis: for C₂₈H₃₂N₄O₄ found (calculated): C, 68.85 (68.83); H, 6.58 (6.60); N, 11.44 (11.47)% |
| 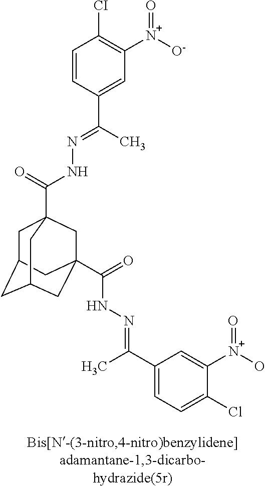 | M.p.: 178 ° C., Yield 66.25 %, Yellow to orange, Mol. Wt. 615.46452 ¹H-NMR (600 MHz, DMSO-d6, δ ppm): 8.54(1H, s, NH—N); 7.93-8.21 (Ar—H); 3.4 (═CH); 1.62-2.00 (Adamantane-H) IR (ν cm⁻¹): 839 (C—Cl); 1074 (N—N); 1247-1296 (C—N); 1511-1513 (C═C benzene); 1533 (NO₂); 1594 (C═N); 1688 (C═O); 2924 (N—H); 3098 (C—H); 3434 (H—C═) Analysis: for C₂₈H₂₈C₁₂N₆O₆ found (calculated): C, 54.62 (54.64); H, 4.56 (4.59); N, 13.63 (13.65)% |

### Example 2

### Antimicrobial Activity Test of the Compounds

The antimicrobial activity of the compounds was determined by tube dilution method. Powdery compounds were taken in closed glass vials and were tested against Staphylococcus aureus (ATCC 6538) (bacteria) and Candida Albicans (ATCC 10231) (Fungi). 50 mg of the sample was taken in 1 ml of DMSO and the contact time for the sample was maintained as 3 minutes. The medium for microorganisms was Soyabean Casein Digest Agar for bacteria and Sabouraud's Dextrose Agar for fungi. The incubation time for bacteria was 2 days at 30-35° C. while for fungi was 3-5 days at 20-25° C. Results of the test are provided in Table 2 below.

TABLE 2
Antimicrobial activity

| | S. aureus | | | C. albicans | | |
|---|---|---|---|---|---|---|
| Sample | Inoculum | Final | % | Inoculum | Final | % |
| Name | count | Count | killing | Count | Count | killing |
| 5a | 19900000 | 0 | 100.00 | 6400000 | 0 | 100.00 |
| 5b | 17400000 | 1680000 | 90.34 | 6400000 | 0 | 100.00 |
| 5c | 17400000 | 2980000 | 82.87 | 7000000 | 10 | 100.00 |
| 5d | 20700000 | 10300000 | 50.24 | 6800000 | 0 | 100.00 |
| 5e | 19700000 | 16400000 | 16.75 | 5900000 | 0 | 100.00 |
| 5f | 20100000 | 16000000 | 20.40 | 6600000 | 0 | 100.00 |
| 5g | 17800000 | 10 | 100.00 | 6700000 | 0 | 100.00 |
| 5h | 18300000 | 0 | 100.00 | 6100000 | 0 | 100.00 |
| 5i | 20800000 | 3120000 | 85.00 | 6800000 | 0 | 100.00 |
| 5j | 19400000 | 550000 | 97.16 | 6600000 | 0 | 100.00 |
| 5k | 18900000 | 7500000 | 60.32 | 6100000 | 0 | 100.00 |
| 5l | 18100000 | 0 | 100.00 | 6900000 | 0 | 100.00 |
| 5m | 18600000 | 1230000 | 93.39 | 7100000 | 0 | 100.00 |
| 5n | 19800000 | 10 | 100.00 | 5800000 | 0 | 100.00 |
| 5o | 20300000 | 18400000 | 9.36 | 6000000 | 0 | 100.00 |
| 5p | 20400000 | 2310000 | 88.68 | 6100000 | 0 | 100.00 |
| 5q | 19500000 | 14400000 | 26.15 | 6500000 | 0 | 100.00 |
| 5r | 19800000 | 5100000 | 74.24 | 6400000 | 0 | 100.00 |

All the samples were found to be anti-microbially active against both organisms. The samples were found to be very effective against Candida albicans and had minimum killing percentage of 100. The samples were also found to be effective against Staphylococcus aureus with varying results and minimum killing percentages of up to 9.36 and 16.75.

The foregoing examples are merely illustrative and are not to be taken as limitations upon the scope of the invention. Various changes and modifications to the disclosed embodiments will be apparent to those skilled in the art. Such changes and modifications may be made without departing from the scope of the invention.

I claim:

1. A compound of Formula I, a stereoisomer, a tautomer, a pharmaceutically acceptable salt or a pharmaceutically acceptable solvate thereof, 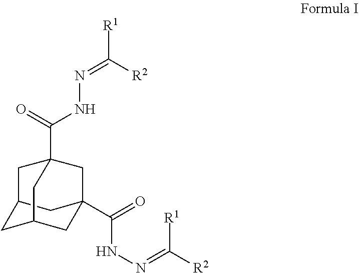
wherein R¹ is an unsubstituted or substituted C₆₋₁₀ aryl or an unsubstituted or substituted C₄₋₁₀ heterocyclyl;
R² is selected from H, C₁₋₆alkyl, C₁₋₆alkoxy, halogen, nitro, —COOH or combinations thereof; and
wherein the substituents are selected from one or more of halogen, nitro, C₁₋₆alkyl, C₁₋₆alkoxy, —COOH, hydroxy, cyano, or NH₂.

2. The compound as claimed in claim 1, wherein R¹ is unsubstituted or substituted phenyl or napthyl and wherein the substituents are selected from one or more of nitro, hydroxy, C₁₋₆ alkoxy, or C₁₋₆alkyl.

3. The compound as claimed in claim 1, wherein R¹ is unsubstituted or substituted C₄₋₁₀ heterocyclyl and wherein the substituents are selected from one or more of nitro, hydroxy, C₁₋₆ alkoxy, or C₁₋₆alkyl.

4. The compound as claimed in claim 1, wherein the C₁₋₆alkoxy is methoxy, or ethoxy.

5. The compound as claimed in claim 1, wherein the C₁₋₆alkyl is methyl.

6. The compound as claimed in claim 1, wherein R² is selected from H or C₁₋₆alkyl.

7. The compound as claimed in claim 1, wherein the compound of Formula I is:
Bis [N′-(4-nitro)benzylidene]adamantane-1,3-dicarbohydrazide;
Bis [N′-(3-methoxy, 4-hydroxy)benzylidene]adamantane-1,3-dicarbohydrazide;
Bis [N′-(1-(2-hydroxy, 5-methyl phenyl)ethylidene)]adamantane-1,3-dicarbohydrazide;
Bis [N′-(3,4-dimethoxy)benzylidene)]adamantane-1,3-dicarbohydrazide;
Bis [N′-(1-(4-ethoxy)ethylidene)]adamantane-1,3-dicarbohydrazide;
Bis [N′-(2-napthyl)methylene)]adamantane-1,3-dicarbohydrazide;
Bis [N′-(5-nitro, furan-2-yl)methylene)]adamantane-1,3-dicarbohydrazide;
Bis [N′-(3-nitro, 4-hydroxy, 5-methoxy) benzylidene]adamantane-1,3-dicarbohydrazide;
Bis[N′-(4-methyl)benzylidene]adamantane-1,3-dicarbohydrazide;
Bis[N′-(4-hydroxy)benzylidene]adamantane-1,3-dicarbohydrazide;
Bis[N′-(1-(4-hydroxyphenyl)ethylidene)]adamantane-1,3-dicarbohydrazide;
Bis[N′-(3-bromo,4hydroxy,5-methoxy)benzylidene]adamantane-1,3-dicarbohydrazide;
Bis[N′-thiophene-2-yl)methylene]adamantane-1,3-dicarbohydrazide;
Bis[N′-(4-ethoxy)benzylidene]adamantane-1,3-dicarbohydrazide;
Bis[N′-(3-nitro,4-chloro)benzylidene]adamantane-1,3-dicarbohydrazide;
Bis[N′-(4-bromo)benzylidene]adamantane-1,3-dicarbohydrazide;
Bis[N′-(4-methoxy)benzylidene]adamantane-1,3-dicarbohydrazide;
Bis[N′-(1-(3-nitro,4-chloro)ethylidene)]adamantane-1,3-dicarbohydrazide;
a stereoisomer, a tautomer, a pharmaceutically acceptable salt or a pharmaceutically acceptable solvate thereof.

8. A pharmaceutical composition comprising a compound of Formula I as claimed in claim 1 and a pharmaceutically acceptable excipient.

9. A process of synthesis of a compound of Formula I, a stereoisomer, a tautomer, a pharmaceutically acceptable salt or a pharmaceutically acceptable solvate thereof comprising the steps of:
(a) reacting a dimethyl-1,3-adamantane-di-carboxylate compound of Formula IV with hydrazine hydrate in the presence of an alcoholic solution to give a dihydrazide compound of Formula III; and 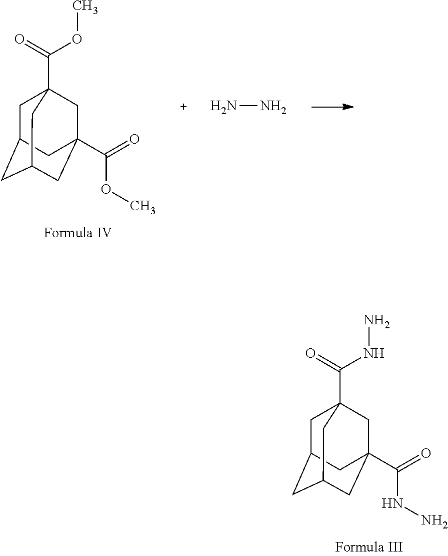
(b) refluxing the compound of Formula III with a compound of Formula II in the presence of a solvent to give the compound of Formula I

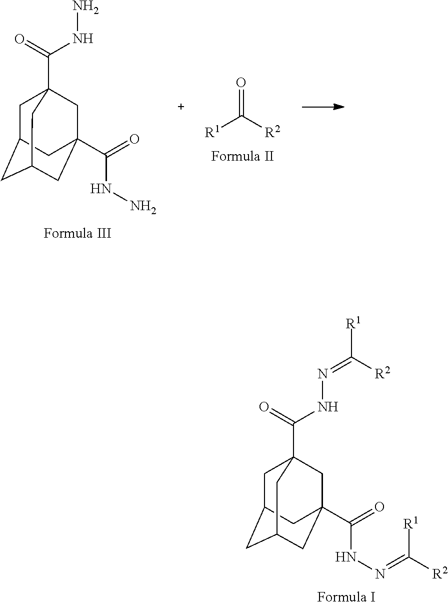

10. The process as claimed in claim 9, wherein the alcoholic solution of step (a) is methanolic or ethanolic solution and the reaction is performed at a temperature range of 0-5° C.

11. The process as claimed in claim 9, wherein the solvent is selected from methanol, ethanol, or combinations thereof.

12. The process as claimed in claim 9, wherein the compound of Formula IV is synthesized by esterification by refluxing a dicarboxylic compound of Formula V with methanol in sulphuric acid

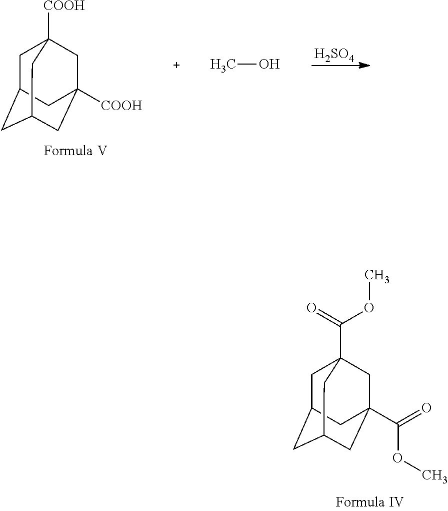

13. A method of inhibiting the growth of a microbe by adding an effective amount of the compound as claimed in claim 1.
